(19) United States
(12) Patent Application Publication
(10) Pub. No.: US 2015/0365738 A1
(43) Pub. Date: Dec. 17, 2015

(54) TELEMETRY ARRANGEMENTS FOR IMPLANTABLE DEVICES

(71) Applicants: Rick Purvis, Pasadena, CA (US); William Andrew Brandt, Castaic, CA (US); Atoosa Lotfi, Valencia, CA (US); Didier Sagan, San Diego, CA (US); Fukang Jiang, Pasadena, CA (US); Greg Harbers, Valencia, CA (US); Brett Daniel Schleicher, New York, NY (US); Nenad Jelisavcic, Altadena, CA (US); Yu-Chong Tai, Pasadena, CA (US); Faysal Ahmed, San Diego, CA (US); Rudy Montalvo, Woodland Hills, CA (US); Tuan Pham, Montclair, CA (US); Steven Arroyo, Padadena, CA (US)

(72) Inventors: Rick Purvis, Pasadena, CA (US); William Andrew Brandt, Castaic, CA (US); Atoosa Lotfi, Valencia, CA (US); Didier Sagan, San Diego, CA (US); Fukang Jiang, Pasadena, CA (US); Greg Harbers, Valencia, CA (US); Brett Daniel Schleicher, New York, NY (US); Nenad Jelisavcic, Altadena, CA (US); Yu-Chong Tai, Pasadena, CA (US); Faysal Ahmed, San Diego, CA (US); Rudy Montalvo, Woodland Hills, CA (US); Tuan Pham, Montclair, CA (US); Steven Arroyo, Padadena, CA (US)

(21) Appl. No.: 14/818,511

(22) Filed: Aug. 5, 2015

Related U.S. Application Data
(63) Continuation-in-part of application No. 14/593,607, filed on Jan. 9, 2015.
(60) Provisional application No. 61/925,318, filed on Jan. 9, 2014.
(60) Provisional application No. 62/033,567, filed on Aug. 5, 2014.

(51) Int. Cl. H04Q 9/00; G08C 17/04; H01Q 7/06; A61F 2/70; A61M 5/142

(52) U.S. Cl. CPC H04Q 9/00; A61F 2/70; A61M 5/14276; H01Q 7/06; G08C 17/04; A61F 2002/701; A61M 2205/3523; A61M 2205/04; H04Q 2209/40

(57) ABSTRACT

In various embodiments, a system for receiving wireless power includes a magnetic core and a plurality of layers of unbraided electrical conductors wrapped around the magnetic core. Each unbraided electrical conductor includes a first endpoint and a second endpoint; the first endpoint and the second endpoint are electrically connected to a circuit to provide power thereto, and two of the unbraided electrical conductors are electrically insulated from each other between the first endpoint and the second endpoint. The system may include a biocompatible, magnetically transparent, rigid polymer casing surrounding the magnetic core and at least a portion of the electrical conductors.

### CROSS-REFERENCES TO RELATED APPLICATIONS

This application is a continuation-in-part of U.S. Ser. No. 14/593,607, filed on Jan. 9, 2015, which claims priority to and the benefit of U.S. Provisional Application No. 61/925,318, filed on Jan. 9, 2014. This application also claims priority to and the benefit of U.S. Provisional Application No. 62/033,567, filed on Aug. 5, 2014. The entire disclosures of these documents are hereby incorporated by reference.

### BACKGROUND

As patients live longer and are diagnosed with chronic and often debilitating ailments, the result will be an increase in the need to place protein therapeutics, small-molecule drugs and other medications into targeted areas throughout the body that are currently inaccessible or inconvenient as sites of administration. For example, many vision-threatening diseases, including retinitis pigmentosa, age-related macular degeneration (AMD), diabetic retinopathy, and glaucoma, are incurable and yet difficult to treat with currently available therapies: oral medications have systemic side effects; topical applications may sting and engender poor compliance; injections require a medical visit, can be painful and risk infection; and sustained-release implants must typically be removed after their supply is exhausted (and offer limited ability to change the dose in response to the clinical picture). Another example is cancer, such as breast cancer or meningiomas, where large doses of highly toxic chemotherapies such as rapamycin or irinotecan (CPT-11) are administered to the patient intravenously, resulting in numerous undesired side effects outside the targeted area.

Implantable drug delivery systems, which may have a refillable drug reservoir, cannula and check valve, etc., allow for controlled delivery of pharmaceutical solutions to a specified target. This approach can minimize the surgical incision needed for implantation and avoids future or repeated invasive surgery or procedures. Refillable ocular drug pumps, for example, usually hold less than 100 μL, are much smaller and more difficult to access post-implantation than other implantable pumps, such as those used for intrathecal injections or insulin therapy.

An implantable drug-delivery pump may incorporate telemetry to facilitate communication with an external monitoring device and wireless charging of the battery powering the implanted device via inductive coupling. In particular, the operating parameters of the implantable pump may be non-invasively adjusted and diagnostic data may be read out from the implantable pump to the external monitoring device through signals transmitted by and received from the telemetry device. During a scheduled visit, a physician may place the monitoring device near the implantable pump and send signals thereto. The implant, in turn, adjusts pump parameters and transmits responses to the monitoring device. By incorporating wireless charging technology, electronic medical implants benefit from a smaller total footprint by reducing the battery size.

Typically, the telemetry circuitry comprises a coil antenna that transmits and receives signals via inductive coupling. A number of parameters characterizing the efficiency of the coil antenna, e.g., the resonant frequency, gain, quality (Q) factor, and thermal effects (i.e., the Joule effect or heat) are considered when selecting or designing the coil antenna. However, the challenges of designing an antenna small enough to fit within an implant while exhibiting adequate performance characteristics, notwithstanding tissue attenuation that weakens the inductive link, are considerable.

### SUMMARY

In various embodiments, the present invention relates to telemetry coils, circuits, and related manufacturing techniques. The present invention is applicable to a wide variety of implanted devices including pacemakers, medicine-delivery pumps, stimulation devices, and artificial hearts. For illustrative purposes, the description that follows will generally focus on the use of the invention within devices having small footprints, e.g., electrolysis-actuated, implantable drug-delivery pumps as described, for example, in U.S. Pat. No. 8,231,608, the entire disclosure of which is hereby incorporated by reference.

In a first aspect, the invention relates to a system for receiving wireless signals. In various embodiments, the system comprises a magnetic core and a plurality of layers of unbraided electrical conductors wrapped around the magnetic core, wherein each unbraided electrical conductor comprises a first endpoint and a second endpoint, the first and second endpoints being electrically connected to a circuit to provide the signal thereto, at least two of the unbraided electrical conductors being electrically insulated from each other between the first endpoint and the second endpoint and interleaved in the first layer.

In some embodiments, the first layer surrounds the magnetic core and a second layer surrounds the first layer, the second layer consisting of a single electrically connected wire. The circuit may, for example, control an implantable medical device. In various embodiments, the second endpoint comprises a tap and another unbraided electrical conductor is electrically connected between the second endpoint and a third endpoint. The unbraided electrical conductors between the first and second endpoints and between the second and third endpoints may receive energy from a primary winding and the unbraided electrical conductors between the first and second endpoints provide the signal to the circuit.

In various embodiments, the system further comprises a biocompatible material adhered to and surrounding the magnetic core and at least a portion of the electrical conductors. The biocompatible material may be, for example, parylene and/or silicone. The system may further comprise a biocompatible, magnetically transparent, rigid polymer casing surrounding the magnetic core and at least a portion of the electrical conductors. For example, the casing may be polysulfone or a ceramic.

In another aspect, the invention relates to a method of operating an implantable medical device including control circuitry and, electrically connected thereto, a telemetry coil comprising a magnetic core and a plurality of layers of unbraided electrical conductors wrapped around the magnetic core, wherein each unbraided electrical conductor comprises a first endpoint and a second endpoint, the first and second endpoints being electrically connected to a the circuitry, at least two of the unbraided electrical conductors being electrically insulated from each other between the first endpoint and the second endpoint and interleaved in the first layer. In various embodiments, the method comprises the steps of wirelessly transmitting, via inductive coupling, a signal to the telemetry coil; and causing the control circuitry to operate the medical device based at least in part on the signal.

In some embodiments, the first layer surrounds the magnetic core and a second layer surrounds the first layer, the second layer consisting of a single electrically connected wire. The second endpoint may comprise a tap and another unbraided electrical conductor may be electrically connected between the second endpoint and a third endpoint. In various embodiments, unbraided electrical conductors between the first and second endpoints and between the second and third endpoints receive energy from a primary winding and the unbraided electrical conductors between the first and second endpoints provide the signal to the circuit. The signal may be a power signal delivering power to the circuit.

In yet another aspect, the invention pertains to a system for receiving wireless signals. In various embodiments, the system comprises a magnetic core having an axial extent and a plurality of layers of electrical conductors wrapped around the magnetic core and extending axially therealong, the electrical conductors each comprising a first endpoint and a second endpoint, the first and second endpoints being electrically connected to a circuit to provide the signal thereto. The electrical conductors are arranged in a sequence of coil elements alternating between first and second configurations along the axial extent. The first and second configurations are mirror images of each other, and each of the coil elements is electrically connected to an adjacent coil element. The system also includes a biocompatible material adhered to and surrounding the magnetic core and at least a portion of the electrical conductors. For example, the biocompatible material may be parylene and/or silicone. The system may further comprise a biocompatible, magnetically transparent, rigid polymer casing surrounding the magnetic core and at least a portion of the electrical conductors. For example, the casing may be polysulfone or a ceramic.

In some embodiments, each of the coil configurations includes an inner open ring and an outer open ring. The inner open ring of a first coil element having the first configuration is electrically connected, across an insulator, to the outer open ring of a second coil element (having the second configuration) adjacent thereto, and the outer open ring of the first coil element is electrically connected, across the insulator, to the inner open ring of the second coil element. Adjacent coil elements may be rotated with respect to each other to accommodate electrical connections therebetween. The circuit may control an implantable medical device.

Yet another aspect of the invention relates to a method of operating an implantable medical device including control circuitry and, electrically connected thereto, a telemetry coil comprising a magnetic core having an axial extent and a plurality of layers of electrical conductors wrapped around the magnetic core and extending axially therealong, the electrical conductors each comprising a first endpoint and a second endpoint, the first and second endpoints being electrically connected to a circuit to provide the signal thereto, wherein the electrical conductors are arranged in a sequence of coil elements alternating between first and second configurations along the axial extent, the first and second configurations being mirror images of each other, each of the coil elements being electrically connected to an adjacent coil element. In various embodiments, the method comprises the steps of wirelessly transmitting, via inductive coupling, a signal to the telemetry coil and causing the control circuitry to operate the medical device based at least in part on the signal.

Each of the coil configurations may include an inner open ring and an outer open ring, wherein (i) the inner open ring of a first coil element having the first configuration is electrically connected, across an insulator, to the outer open ring of a second coil element adjacent thereto, the second coil element having the second configuration, and (ii) the outer open ring of the first coil element is electrically connected, across the insulator, to the inner open ring of the second coil element. Adjacent coil elements may be rotated with respect to each other to accommodate electrical connections therebetween.

Still another aspect of the invention relates to a method for manufacturing a coil. In various embodiments, method comprises creating a first polymer layer on a substrate; depositing a metal layer on the first polymer layer; patterning the first metal layer to form inner and outer conducting coils; depositing a second polymer layer on the patterned first metal layer; patterning the second polymer layer to open a metal-interconnection portion of the first metal layer; and depositing a second metal layer on the patterned second polymer layer, wherein the second metal layer contacts the opened metal interconnection portions of the first metal layer.

Yet another aspect relates to drug-delivery system comprising, in various embodiments, a hermetically sealed enclosure including control circuitry; a discrete drug-dispensing assembly comprising a drug reservoir and a pumping mechanism; and a telemetry coil disposed within a discrete, magnetically transparent casing. The drug-dispensing assembly and the casing are separately joined to the enclosure mechanically and with electrical connection to the control circuitry, whereby the control circuitry receives power and/or control signals via the telemetry coil and selectively actuates the drug-dispensing assembly in accordance with a drug-delivery protocol. For example, the pumping mechanism may be electrolytic, e.g., using electrolysis electrodes to evolve a gas that drives drug from a drug chamber or reservoir.

The term “substantially” or “approximately” means±10% (e.g., by weight or by volume), and in some embodiments, ±5%. The term “consists essentially of” means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

The foregoing will be more readily understood from the following detailed description of the invention, in particular, when taken in conjunction with the drawings, in which:

FIG. 1 illustrates a system that includes a drug-delivery pump in accordance with embodiments of the present invention;

FIGS. 2A and 2B illustrate a coil having wires wrapped in a pseudo-Litz configuration in accordance with embodiments of the present invention;

FIGS. 2C and 2D illustrate encasement of the coil shown in FIGS. 2A and 2B;

FIGS. 2E and 2F illustrate deployment of an encased coil in an implantable drug pump;

FIG. 3 illustrates a schematic diagram of an existing primary/secondary coil telemetry system;

FIG. 4 illustrates a schematic diagram of a voltage divider circuit in accordance with embodiments of the present invention;

FIG. 5 illustrates current-voltage curves of the performance of embodiments of the present invention;

FIGS. 6A and 6B illustrate cross-sectional views of coil layers in accordance with embodiments of the present invention;

FIG. 7 illustrates a three-dimensional view of coil layers in accordance with embodiments of the present invention;

FIG. 8 illustrates a perspective view of a coil layer in accordance with embodiments of the present invention; and

FIGS. 9A and 9B illustrate three-dimensional views of coil layers in accordance with embodiments of the present invention.

### DETAILED DESCRIPTION

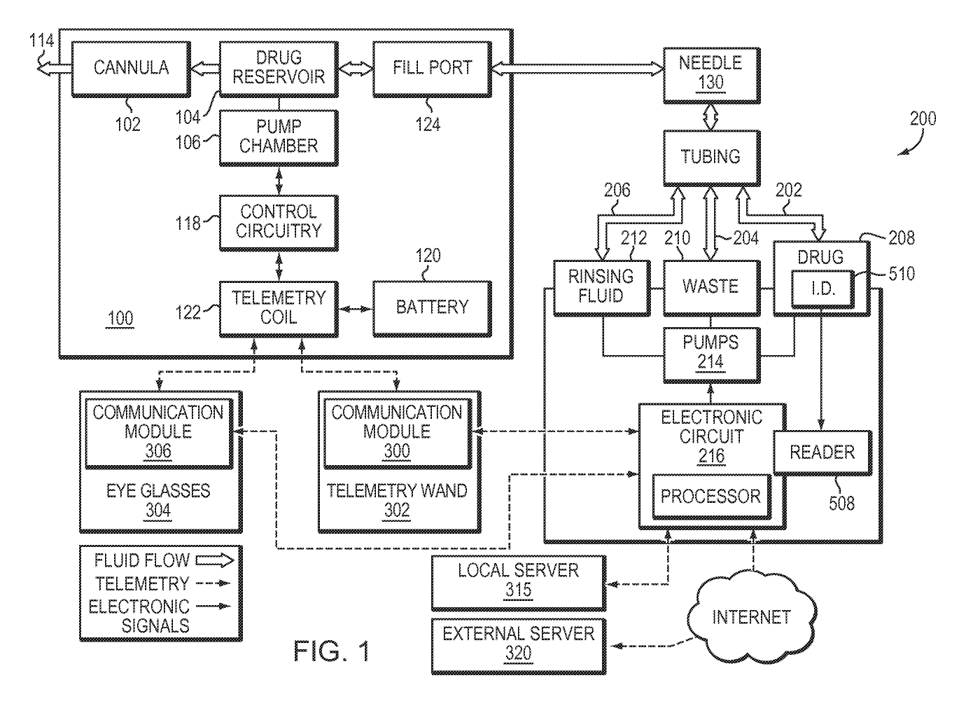

FIG. 1 illustrates an exemplary system involving a drug-delivery pump 100. The drug pump device 100 includes a cannula 102 and a pair of chambers 104, 106. The top chamber 104 defines a drug reservoir that contains the drug to be administered in liquid form, and the bottom chamber 106 contains a liquid which, when subjected to electrolysis using electrolysis electrodes 110, evolves a gaseous product. The two chambers are separated by a corrugated diaphragm (not shown). The cannula 102 connects the top drug chamber 104 with a check valve 114 inserted at the site of administration. Control circuitry 118, a battery 120, and an induction coil 122 for power and data transmission are embedded within the device 100. Depending on the complexity of the control functionality it provides, the control circuitry 118 may be implemented, e.g., in the form of analog circuits, digital integrated circuits (such as, e.g., microcontrollers), or programmable logic devices. In some embodiments, the control circuitry 118 includes a microprocessor and associated memory for implementing complex drug-delivery protocols. The drug pump device 100 may also include various sensors (e.g., pressure and flow sensors) for monitoring the status and operation of the various device components, and such data may be logged in the memory for subsequent retrieval and review.

Importantly for the prolonged use of the drug pump device 100 following implantation, the device 100 includes one or more fill ports 124 in fluid communication with the drug reservoir 104, which permit a refill needle (not shown) to be inserted therethrough. The refill port 124 may define an aperture through the wall of the reservoir 104, which may be closed and sealed with a septum or plug made of a puncturable, self-sealing material, allowing a non-coring needle 130 (e.g., a needle that does not remove any of the material it punctures) to pierce through the septum while ensuring that the septum reseals itself, or “heals,” upon removal of the needle. Preferably, the self-sealing material is biocompatible and able to withstand multiple punctures by the needle. The septum or plug may be made of any of a variety of elastomeric polymers (such as, e.g., silicone, polydimethylsiloxane (PDMS), polyurethane, polyethylene, parylene C, or rubber), and the specific composition of the polymer mixtures may be chosen so as to enhance the self-sealing properties. Silicone, for example, is naturally self-healing, but this property is more pronounced in particular formulations well-known to persons of skill in the art. The septum material may be injected directly into the aperture of the port and cured in place. In some embodiments, a slit is pre-formed in the septum, and the needle is inserted along this slit; the septum and surrounding port walls are sized such that radial pressure from the walls of the port compresses the septum and, with it, the slit, preventing leakage during filling and after the needle has been removed.

Through the refill port 124, the existing fluid in the reservoir (such as any residual drug) can be removed, the reservoir washed, and a filling/refilling solution injected. Certain embodiments of the invention involve an external refill system 200 that can be interfaced to the drug reservoir for the automatic filling/refilling of the reservoir 104. The refill system 200 generally includes at least two channels: one channel 202 for aspirating fluid (e.g., expired and/or remnant drug) from the reservoir of the drug pump device 100 and another channel 204 for loading new drug into the reservoir. In some embodiments, the system further includes a third channel 206 for rinsing the drug pump prior to filling it with the fresh drug. Each channel is fluidically connectable to a depot or container, e.g., one containing the drug to be dispensed (208), one receiving waste liquid from the drug pump device 100 (210), and one containing the rinsing solution (212). This rinsing solution may be the drug vehicle (e.g., a fluid missing the active ingredient, but otherwise having the same composition as the liquid drug), or any solution compatible with the drug and drug pump device 100.

The refill system 200 also includes one or more pumps 214 for generating positive or negative pressure to effect the infusion and suction of liquid into and out of the drug pump device 100. The pumps 214 may be standard mechanical pumps (e.g., gear, diaphragm, peristaltic, or syringe pumps), or pneumatic systems such as, e.g., vacuum generators, air compressors, pneumatic motors, pneumatic actuators, etc. In some embodiments, pressure sensors, flow sensors, and/or valves are integrated into the channels 202, 204, 206 and/or the pumps 214 to facilitate monitoring of the flow rate and/or pressure during the refilling process and controlling pump operation based thereon. The refill system 200 further includes electronic control circuitry 216 that directs the operation of the pump(s), and/or a user interface that allows a user (e.g., a physician or nurse) to provide input to the control circuitry 216 and/or to manually trigger certain pre-defined pump functions (e.g., via buttons, a foot pedal, and/or a conventional computer user interface including, e.g., a screen, keyboard, and mouse). The electronic control circuitry is conventional and typically comprises a processor for performing computations related to the pump operation. The processor may be a general-purpose or special-purpose processor, and may utilize any of a wide variety of data-processing and control technologies, including, e.g., a microcomputer, mini-computer, mainframe computer, programmed microprocessor, microcontroller, peripheral integrated circuit element, CSIC (customer-specific integrated circuit), ASIC (application-specific integrated circuit), logic circuit, digital signal processor, programmable logic device such as an FPGA (field-programmable gate array) or PLA (programmable logic array), RFID processor, or smart chip.

The refill system 200 may be implemented as a single unit or, alternatively, as multiple components. In certain embodiments, the pumps 214, control circuitry 216, and (optionally) valves and sensors are integrated into a reusable base unit, whereas the fluid channels 202, 204, 206 are provided in a replaceable and/or disposable tubing set connectable to the base unit and, at the other end, to the refill needle 130.

In some embodiments, one or more of the containers 208, 210, 212 holding the drug, waste liquid, and rinsing solution are provided in the form of vials or cartridges (hereinafter used synonymously). The waste-liquid cartridge 210 (or other container) may contain a dye that changes the color of the waste liquid upon contact to a noxious or at least noticeably anomalous hue such as black so as to prevent users from inadvertently re-injecting waste drug back into the patient or pump. The dye may, e.g., consist of natural or synthetic dyestuffs that are contained in the cartridge in powder form or line the surface of the cartridge. Furthermore, the cartridge 310 may contain reactive agents that disable use of the drug by destroying its activity, e.g., via an acid-base reaction, but which are non-toxic so as to avoid harm to the patient should the mixture be re-injected.

The refill system 200 may have receiving wells or other receptacles for the cartridges 208, 210, 212. In certain embodiments, the cartridges have a proprietary shape that must mate with a complementary receiving well in the refill system. This approach can also facilitate mechanical locking of the cartridge to the drug refill system, e.g., so that it snaps into place. Mechanical locking may be accomplished, e.g., using a trapezoid, triangle, or hexagonal male connector on the drug refill system and a geometrically complementary connector on the cartridge. Using cartridges of a particular shape in conjunction with matching receiving wells may serve to prevent non-proprietary cartridges or drugs from being used with the refill system 200, e.g., to ensure the integrity of the drug. A further level of security may be obtained by facilitating electronic communication between the cartridge and the refill system 200. For example, the cartridge may have a barcode encoding the identity of the drug therein, and the refill system 200 may be equipped to read the barcode once the cartridge is introduced. Alternatively, the cartridge may have an optical, RF, or similar ID tag or other electronic information storage (e.g., a ROM or an EPROM) that specifies the contents of the cartridge, and which is interrogated by the refill system 200.

In various embodiments, the refill system 200 facilitates wireless communication with the drug pump device 100. For example, the control circuitry 216 unit may include a radio-frequency (RF) transceiver or similar component that exchanges data with the induction coil 122 embedded in the pump device 100. In some embodiments, a communication or telemetry module 300 (including a transceiver and related circuitry) is provided separately from the control circuitry 216, e.g., in a handheld telemetry wand 302 that allows the operator (e.g., a nurse or physician) to conveniently bring the wand in the vicinity of the implanted pump device 100, thereby reducing power requirements on the device 100 and, consequently, its footprint. The wand may be corded to the refill system 200 or may communicate therewith via a separate wireless connection. In some embodiments, special eyeglasses 304 equipped with a telemetry module 306 are used to recharge the pump's battery 120 via the induction coil 122; such eyeglasses are described in in the '608 patent mentioned above. These eyeglasses 304 and the refill system 200 may be connected to each other or to a common console, and wireless data exchange with the drug pump device 100 may occur via the eyeglasses rather than a separate telemetry wand 302.

Via the telemetry module (of the telemetry wand 502 or the eyeglasses 504), the base unit may send refill information, including, e.g., the type of drug, the volume injected into the reservoir, a drug dosing schedule, and the date of refill, to the pump device 100. The drug pump device 100 may store this information in its on-board memory, preferably in encrypted form to ensure patient privacy, and may provide it when later interrogated by the refill system or other wireless device. The previous refill information may be used to ensure that the refill drug—as determined by the refill system's electronic label reader 508 from the barcode, RFID, optical ID, EPROM, or other electronic label 510 of the cartridge, or from the proprietary shape of the cartridge—matches the previously administered drug, thereby preventing off-label or other improper uses of the pump. Alternatively or additionally, the pump device 100 may be programmed to accept only a particular drug, and when wireless communication is established between the refill system 300 and the pump device 100, the refill system 300 and the pump exchange information to ensure that the refill drug matches the drug for which the pump was programmed. In either embodiment, refill is prevented—typically by disabling operation of the refill system—if a match is not registered. Of course, it may sometimes be necessary or desirable to change the drug administered to the patient, e.g., if a previously used drug caused complications. In this case, the operator may override the control signal that prevents the refill from commencing and/or reprogram the drug pump device 100 for the new drug.

The communication link established between the drug pump device 100 and the refill system 200 may also be used to download a stored drug dosing log or the pump operation history (including, e.g., information about any error conditions that have occurred in the pump device 100 since the previous communication) from the drug pump device 100. This information may be displayed on a screen (e.g., of a computer console that is part of the user interface) prior to commencement of the refill procedure, enabling the physician to detect any problems with the operation of the device 100 and relate the patient's condition to drug administration over extended periods of time. The physician may, for example, have the option to display twelve months of pump history or ten years of drug delivery history. Further, the physician may reprogram the drug pump device 100 at the time of refill, e.g., to adjust dosage protocols in response to changes in the patient's condition or new insights derived from medical research. In some embodiments, communication between the drug pump device 100 and the refill system 200 is sustained during the refill procedure to facilitate monitoring the process based on sensor readings acquired in the pump device 100. For example, sensors may continuously measure the pressure and fill stage of the drug reservoir 104, and send this data to the refill system 200, where it provides feedback to the processor of the control circuitry 216 and/or a physician manually controlling the refill system 200.

The data exchanged with the drug pump device 100 may be stored on a local server 315 integrated with or connected to the drug refill system 200. Alternatively, the communication module may permit the refill system 300 to communicate with an external server 320, e.g., remotely via the Internet. For example, the refill system 200 may have Wi-Fi, Zigbee, or a cellular phone chip (GSM, CDMA) that is constantly activated to cellular service or other wireless capability. This permits patient and drug data to be stored outside the refill system 300 (“in the cloud”), and may provide further levels of security and operational flexibility.

As noted earlier, the design of the telemetry coil 122 can present significant challenges, including overcoming the “skin effect” (i.e., a high current density only near the surface or “skin” of the coil conductors at high frequencies) that attenuates energy collected thereby. One approach to mitigating the skin effect is a type of cable called Litz wire, which is a braided or stranded wire that minimizes AC impedance by maximizing the conductive cross-section at a given operating frequency; as a result, Litz wire reduces skin-effect losses, and may be wrapped around a magnetic material (e.g., a ferrite core) to create an inductive coil with increased magnetic flux density. Litz wire typically consists of multiple wire strands insulated electrically from each other. Unfortunately, conventional Litz wire using a braided configuration is typically too large for use in many implant devices. For example, in a representative ocular implant, the height size restriction is less than 150 m including wire insulation. Furthermore, some wire gauges are unsuitable as the benefit of the Litz wire in reducing the skin effect disappears once the skin depth is greater than the radius of the wire in accordance with the following equation:

$$δ = \sqrt{\frac{2}{ω μ σ}}$$

where δ is the skin depth, ω is the angular frequency of the current, μ is the material permeability, and σ is the material conductivity.

In conventional Litz wire, the strands are insulated from each other (otherwise all the wires in the bundle would short together, behave like a single large wire, and thereby fail to mitigate the skin effect). Furthermore, the strands cannot occupy the same radial position in a bundle, since the electromagnetic effects that cause the skin effect would otherwise still disrupt conduction. The weaving or twisting pattern of the Litz wire is designed so that the individual strands are on the outside of the bundle for a distance (where the electromagnetic field changes are smaller and the strand sees low resistance), and are inside for a distance (where the field changes are the strongest and the resistance is higher). If each strand has a comparable impedance, current is distributed equally among every strand within the cable.

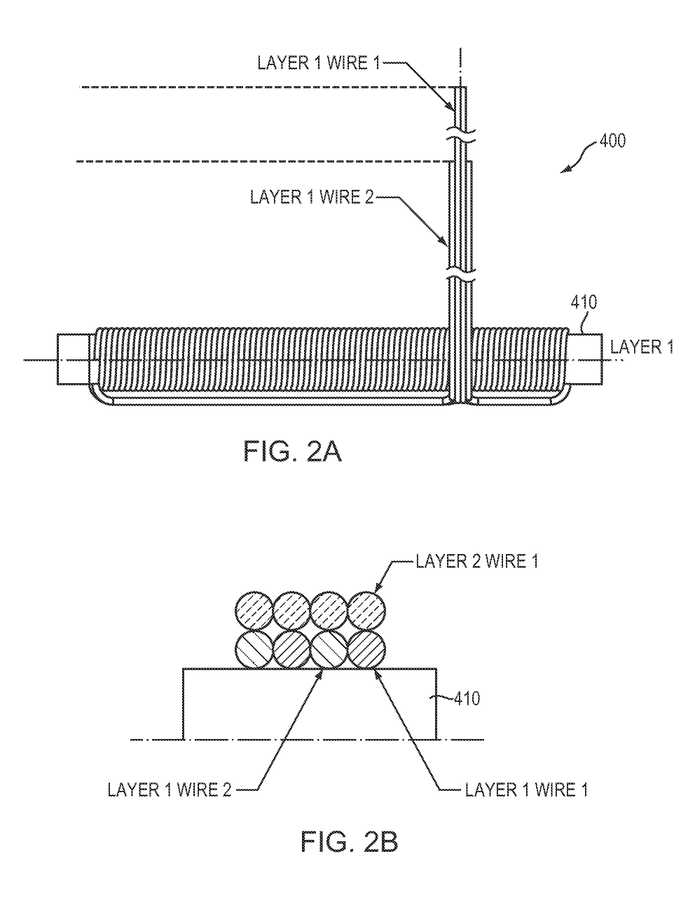

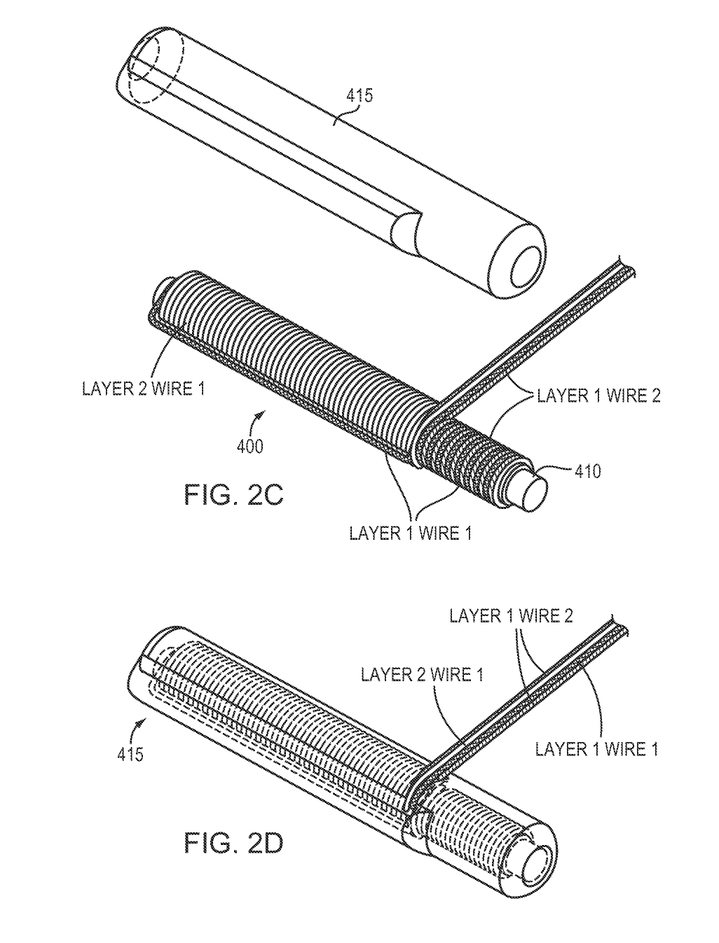

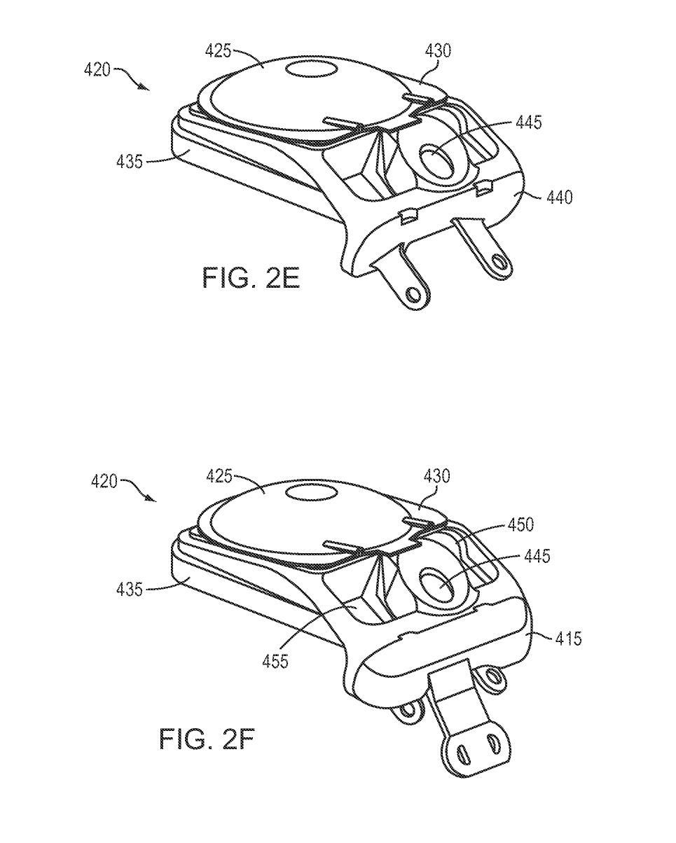

FIGS. 2A and 2B illustrate a substitute for Litz wire in accordance with an embodiment of the present invention; these are sometimes referred to herein as “psedo-Litz” wires in that they address the skin effect without the cumbersome configuration of a traditional Litz wire. Thus, rather than a bundle, the conductor 400 utilizes two or more wire strands (wire 1 and wire 2 in the figure) in parallel that are wound side-by-side, in a single layer, around a magnetic core 410 such that the strands are of equal length; that is, as shown in FIG. 2B, wire 1 and wire 2 alternate in a single layer (layer 1). A second layer consisting of as few as a single wire (layer 2, wire 1) is wound around layer 1, wires 1 and 2. Each of the wires is insulated and has a diameter less than its skin depth at a desired operating frequency or range of frequencies. It is found that this pattern of winding mitigates or avoids skin-effect losses compared to single-core winding without the need for elaborate Litz braiding that would make the wire too thick to use in an implant.

The telemetry coil described above may be made of, or include, materials that are not biocompatible. Therefore, it may be desirable to encapsulate the coil within a biocompatible material for safe long-term implantation. In one approach, the conductors 400 and the core 410 are fully encapsulated in a biocompatible polymeric material, such as parylene (i.e., poly(p-xylylene)), e.g., parylene C. The polymer may be deposited or otherwise applied (e.g., by spraying) over the entire telemetry coil or, if the conductors 400 are already coated in a polymer, over just the core 410. In some embodiments, the telemetry coil is then dipped in a second biocompatible polymer, such as polyurethane, silicone or a biocompatible epoxy (e.g., a HYSOL adhesive such as LOCTITE, available from Henkel Corporation), for greater exterior durability. Alternatively, the conductors 400 and/or the core 410 may be dipped in or sprayed with silicone and then coated with parylene, so that the parylene is presented at the surface. If only the core 410 is to receive a coating, it may be, for example, glass or other hard material more easily applied to a smooth, unitary structure than to a coil. Particularly if the telemetry coil receives an outer coating of a durable biocompatible material, it is not even necessary for an interior coating (if applied) to be biocompatible; for example, the core may be encapsulated in an inert material such as Al₂O₃ or TnO₂ using, for example, atomic layer deposition. Other materials include but are not limited to ceramics that may be selected for biocompatibility and known brazing techniques for encapsulation of the coil.

In other embodiments, as illustrated in FIGS. 2C and 2D, the coil is enclosed in a polymeric casing 415, which may be fabricated from a biocompatible, magnetically transparent, rigid polymer such as polysulfone. The casing 415 may be machined or cast to permit egress of the conductors 400 and/or to contain additional components.

Deployment of an encased coil in a drug-delivery pump is illustrated in FIGS. 2E and 2F. The device 420 typically has a small footprint and its functional components are described, for example, in U.S. Pat. No. 8,231,608, the disclosure of which is hereby incorporated by reference. The drug reservoir is created between the dome-shaped top member 425 and a bottom member 430 that includes a flexible diaphragm; in particular, the top and bottom members 425, 430 are along a peripheral edge, and the flexible diaphragm constitutes the floor of the drug reservoir. Beneath this floor, an electrolysis chamber contains an electrolysis fluid and electrodes which, when actuated, evolve gas as noted above to drive drug from the drug chamber. The drug-dispensing assembly that defines the drug and electrolysis chambers are mounted to a hermetically sealed housing 435, which contains the control circuitry noted above and described in the '608 patent. Mounting of the drug-dispensing assembly secures it mechanically to the housing 435 and establishes electrical connection between the electrodes and the control circuitry via electrical contacts (not shown) without disturbing the hermetic seal of the housing 435. The housing 435 also includes a front face 440 to which the coil-containing casing 415 is mounted. Once again, mounting of the casing 415 to the front face 440 establishes electrical connection between the coil and the hermetically sealed control circuitry via mating electrically contacts as well as mechanical affixation to the face 440. Thus, the coil is retained within a magnetically transparent casing that is directly exposed to the site of implantation for maximum coupling. The housing 435 also includes a refill port 445 that permits the drug chamber to be refilled. Drug may be dispensed through a port in the top member 425 or elsewhere in the pump 420. A pair of wells 450, 455 may receive additional components such as flow or pressure sensors. For example, the drug-delivery path may pass through one of the wells 450, 455. The drug-delivery path may alternatively travel through a portion of the casing 415.

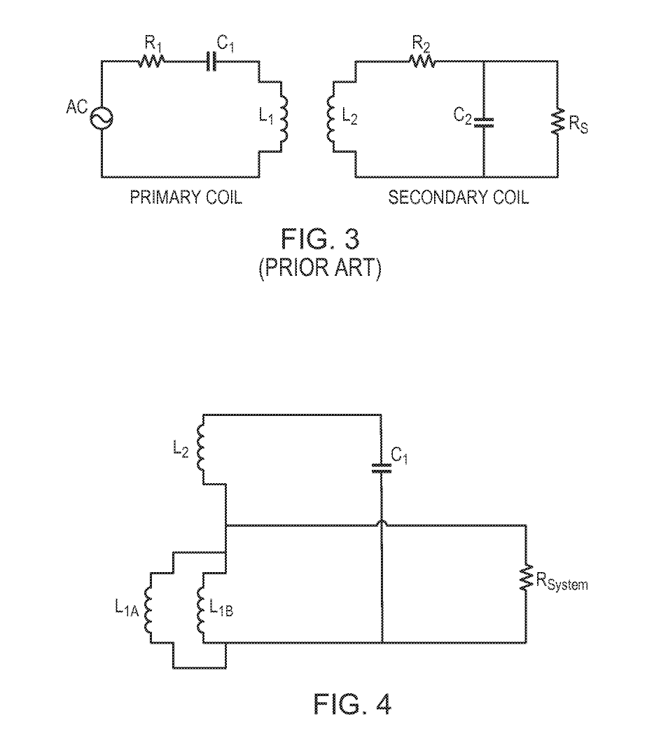

The winding pattern illustrated in FIGS. 2A-2D affords use of an innovative voltage-divider circuit in control circuitry 118. In a conventional circuit, shown in FIG. 3, a primary coil (which would be deployed in the telemetry wand 302 and/or eyeglasses 304) includes an AC source, a capacitor C₁, and an inductor L₁. R₁ denotes the resistive loss in L₁. The secondary coil (which would be deployed in the implant 100) includes an inductor L₂ (which would serve as the telemetry coil 122), a capacitor C₂, and R₃ (the load of the system). R₂ denotes the resistive loss in L₂.

In the alternative secondary-coil circuit 500 shown in FIG. 4, the two first-layer wires of the arrangement shown in FIGS. 2A and 2B form two parallel coils L_{1A}, L_{1B}. The layer 2 winding forms a third coil L₂ in series with the parallel coils L_{1A}, L_{1B}. A tap is taken between the parallel coils L_{1A}, L_{1B} and the third coil L₂ and run through the circuitry 118—that is, a communication module if the signal is modulated to convey information, or the battery 120 if the signal is used to recharge it—which behaves as a load R_{system}. The voltage across the system load is dictated by the number of turns in L₂ relative to L_{1A}, L_{1B}, which can of course be set for a given application. The tap point may be placed at either end of the core 410 or anywhere therealong.

In the circuit 500, the entire coil (L_{1A}, Lᵢₙ, and L₂) contributes to the resonance of the circuit, allowing for a higher loaded Q factor (maximization of self-inductance and low series resistance). However, the implant's circuitry R_{system} is subjected to the lower voltage dictated by the tap, so that circuitry 118 benefits from lower impedance. Despite the voltage division that results in a voltage drop of R_{system} unloaded, it is largely compensated by the resonant voltage increase. It results in a gain as the loaded Q is maintained.

In one implementation, the wire arrangement 400 and the circuit 500 were used to obtain current-voltage (IV) parameters suitable for operation of a particular implantable device. There is a minimum operating voltage as well as a maximum operating voltage as defined by the load as well as a maximum load current.

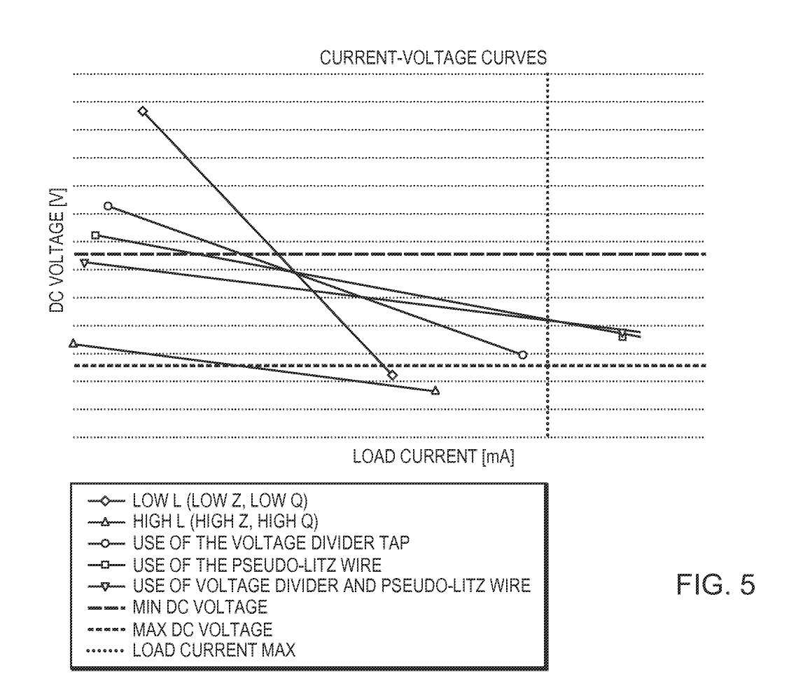

Conventional coils with sizes constrained by volume limitations impose a compromise among inductance, Q factor and impedance. These characteristics can be difficult to match to a particular device or application. Using the present approach, the IV curve fits within the operating parameters of the device. In FIG. 5, the IV curves produced from different combinations are compared, and demonstrate that judicious selection of the conductor material, core dimensions, number and configurations of windings, winding topology, and tap position, a telemetry coil with a high Q factor and advantageous loaded IV characteristics may be created.

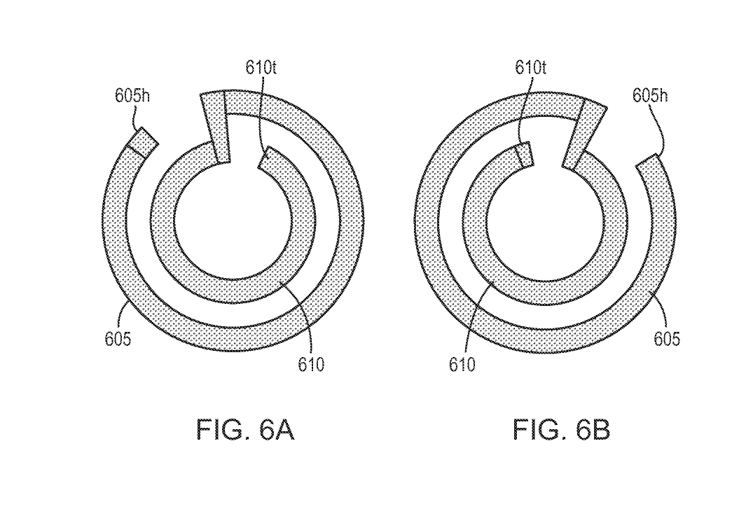

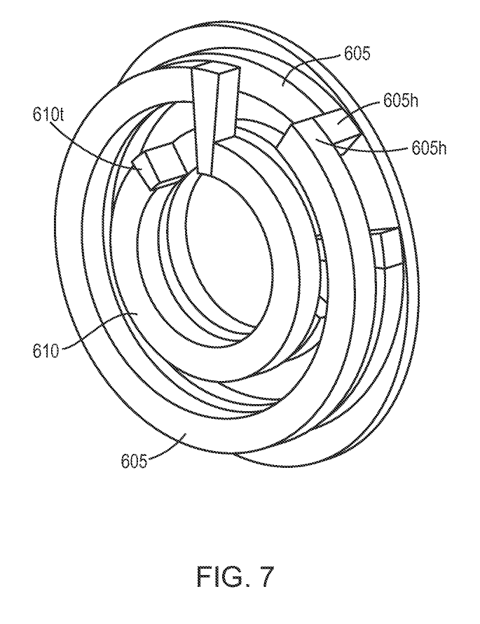

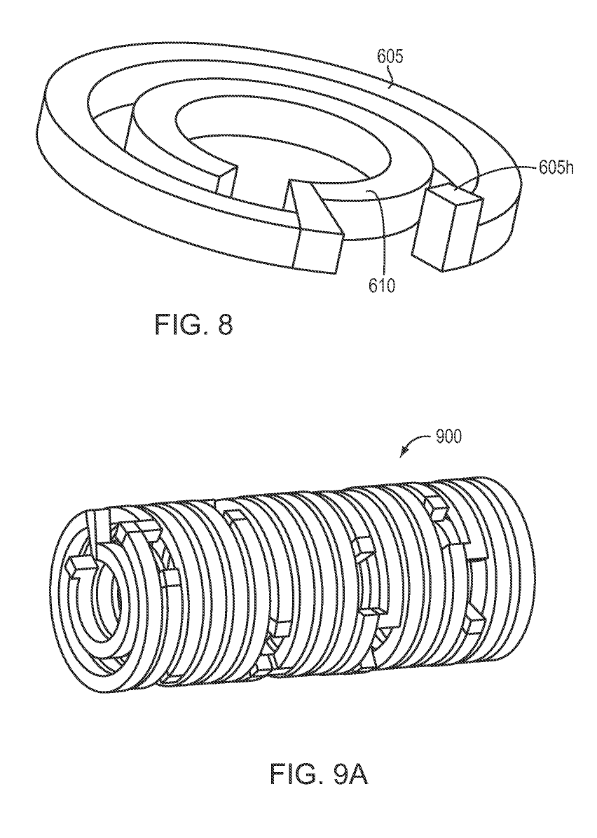

Another approach to skin-effect mitigation uses, instead of wires, conductive traces that may be printed or otherwise deposited onto a substrate. The basic pattern 600 is illustrated in FIGS. 6A, 6B, and 7, and comprises or consists of a series of interconnected outer ring elements 605 and inner ring elements 610. FIGS. 6A and 6B illustrate coil elements 600a, 600b that are mirror images of each other and serve as adjacent coil layers in a sequence of such layers; the coil layers alternate between the two mirror-opposite configurations 600a, 600b along an axis (e.g., surrounding an axial magnetic core). Each layer 600 comprises or consists of an outer ring element, which has a head end 605h in the form of a tab, and an inner ring having a tail end 610t. With reference to FIGS. 7 and 8, an insulating disk 615 intervenes between adjacent coil layers, and the coil elements 600a, 600b are connected across (i.e., traverse) the disk 615 so that the proximal coil element 600b on a first side of the insulating disk 615 electrically connects thereacross to the distal coil element 600a on the opposite side of the disk 615. Similarly, for the next connection, the distal coil element 600a electrically connects, across another insulating disk, to a coil element 600b. FIGS. 7 and 8 illustrate the three-dimensional configuration of the coil elements 600 and how the head and/or tail ends terminate in transversely extending tabs that may be connected to an adjacent coil element through the insulating disk. A tab may be connected to the tab of an adjacent coil element or to a flat head or tail portion thereof.

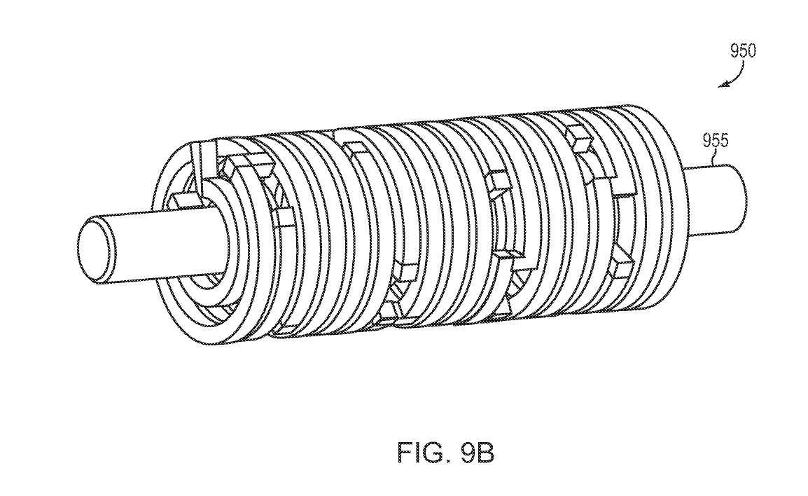

Alternatively, if creation of internal vias to facilitate the interconnection is difficult, interconnects may extend radially over the edge of the disk 615. In that case (as shown in FIG. 9A), when this pattern is extended axially to create a coil 950 consisting of a linear series of coil elements, the head and tail ends shift circumferential positions the by a preset angle along the axial extent of the resulting coil structure 950. This preset angle is ideally minimized to increase the representative equivalent of “coil winding length” without compromising the insulative properties between coil elements. FIG. 9B illustrates that the coil structure 950 can surround a magnetic (e.g., ferrite) core 955, which serves to increase the magnetic field and thus the inductance. The resulting coil structure 950 may be further encapsulated in an epoxy, resin, and/or glass tubing to completely insulate it for use in an implantable medical device.

The arrangements 900, 950 have various advantages compared with conventional Litz wire. First, they may contain more wire turns or coils in a compact form, as insulation coatings surrounding wires are unnecessary. A second advantage is that the minimal interconnect thickness may allow for more coils over a given axial length. A third advantage is the convenience, reproducibility and accuracy achievable with deposition techniques.

Pseudo-Litz wires in accordance herewith may be created in layers using microelectromechanical (MEMS) fabrication technology. A representative sequence of fabrication steps includes or consists of creating a first polymer layer on a substrate; depositing a metal layer on the first (polymer) layer; patterning the first metal layer to form the inner and outer helices; depositing a second polymer layer on the patterned first metal layer; patterning the second polymer layer to open the metal interconnection portion of the first metal layer; and depositing a second metal layer on the patterned second polymer layer, so that the second metal layer contacts the opened metal interconnection portions of the first metal layer. These steps are repeated until the desired configuration is reached. The telemetry coil may be completed in sections, with multiple sections combined to create the desired coil size. The metal may comprise, consist, or consist essentially of gold, copper, or any conductive metal. The polymer layer may be polyimide, parylene, polyesters, polydimethylsiloxane (PDMS) or other non-conductive materials.

Alternatively, pseudo-Litz wires may be created in layers using PCB or Flex technology. For example, traces may be deposited or printed (e.g., by screen printing) onto a flexible substrate such as polyimide, or may be applied using photolithography, or coil layers may be laminated between substrate sheets.

Having described certain embodiments of the invention, it will be apparent to those of ordinary skill in the art that other embodiments incorporating the concepts disclosed herein may be used without departing from the spirit and scope of the invention. For example, various features described with respect to one particular device type and configuration may be implemented in other types of devices and alternative device configurations as well. Accordingly, the described embodiments are to be considered in all respects as only illustrative and not restrictive.

What is claimed is:

1. A system for receiving wireless signals, the system comprising:
a magnetic core; and
a plurality of layers of unbraided electrical conductors wrapped around the magnetic core, wherein each unbraided electrical conductor comprises a first endpoint and a second endpoint, the first and second endpoints being electrically connected to a circuit to provide the signal thereto, at least two of the unbraided electrical conductors being electrically insulated from each other between the first endpoint and the second endpoint and interleaved in the first layer.

2. The system of claim 1, wherein the first layer surrounds the magnetic core and a second layer surrounds the first layer, the second layer consisting of a single electrically connected wire.

3. The system of claim 1, wherein the circuit controls an implantable medical device.

4. The system of claim 1, wherein (i) the second endpoint comprises a tap and (ii) another unbraided electrical conductor is electrically connected between the second endpoint and a third endpoint.

5. The system of claim 4, wherein unbraided electrical conductors between the first and second endpoints and between the second and third endpoints receive energy from a primary winding and the unbraided electrical conductors between the first and second endpoints provide the signal to the circuit.

6. The system of claim 1, further comprising a biocompatible material adhered to and surrounding the magnetic core and at least a portion of the electrical conductors.

7. The system of claim 6, wherein the biocompatible material is parylene.

8. The system of claim 6, wherein the biocompatible material is silicone.

9. The system of claim 6, further comprising a biocompatible, magnetically transparent, rigid polymer casing surrounding the magnetic core and at least a portion of the electrical conductors.

10. The system of claim 9, wherein the casing is polysulfone.

11. The system of claim 10, wherein the casing is ceramic.

12. A method of operating an implantable medical device including control circuitry and, electrically connected thereto, a telemetry coil comprising a magnetic core and a plurality of layers of unbraided electrical conductors wrapped around the magnetic core, wherein each unbraided electrical conductor comprises a first endpoint and a second endpoint, the first and second endpoints being electrically connected to a the circuitry, at least two of the unbraided electrical conductors being electrically insulated from each other between the first endpoint and the second endpoint and interleaved in the first layer, the method comprising the steps of:
wirelessly transmitting, via inductive coupling, a signal to the telemetry coil; and
causing the control circuitry to operate the medical device based at least in part on the signal.

13. The method of claim 11, wherein the first layer surrounds the magnetic core and a second layer surrounds the first layer, the second layer consisting of a single electrically connected wire.

14. The method of claim 11, wherein (i) the second endpoint comprises a tap and (ii) another unbraided electrical conductor is electrically connected between the second endpoint and a third endpoint.

15. The method of claim 11, wherein unbraided electrical conductors between the first and second endpoints and between the second and third endpoints receive energy from a primary winding and the unbraided electrical conductors between the first and second endpoints provide the signal to the circuit.

16. The method of claim 11, wherein the signal is a power signal delivering power to the circuit.

17. A system for receiving wireless signals, the system comprising:
a magnetic core having an axial extent;
a plurality of layers of electrical conductors wrapped around the magnetic core and extending axially therealong, the electrical conductors each comprising a first endpoint and a second endpoint, the first and second endpoints being electrically connected to a circuit to provide the signal thereto, wherein the electrical conductors are arranged in a sequence of coil elements alternating between first and second configurations along the axial extent, the first and second configurations being mirror images of each other, each of the coil elements being electrically connected to an adjacent coil element; and
a biocompatible material adhered to and surrounding the magnetic core and at least a portion of the electrical conductors

18. The system of claim 17 wherein each of the coil configurations includes an inner open ring and an outer open ring, wherein (i) the inner open ring of a first coil element having the first configuration is electrically connected, across an insulator, to the outer open ring of a second coil element adjacent thereto, the second coil element having the second configuration, and (ii) the outer open ring of the first coil element is electrically connected, across the insulator, to the inner open ring of the second coil element.

19. The system of claim 18, wherein adjacent coil elements are rotated with respect to each other to accommodate electrical connections therebetween.

20. The system of claim 17, wherein the biocompatible material is parylene.

21. The system of claim 17, wherein the biocompatible material is silicone.

22. The system of claim 17, further comprising a biocompatible, magnetically transparent, rigid polymer casing surrounding the magnetic core and at least a portion of the electrical conductors.

23. The system of claim 17, wherein the casing is polysulfone.

24. The system of claim 17, wherein the casing is ceramic.

25. A drug-delivery system comprising:
a hermetically sealed enclosure including control circuitry;
a discrete drug-dispensing assembly comprising a drug reservoir and a pumping mechanism; and
a telemetry coil disposed within a discrete, magnetically transparent casing,
wherein the drug-dispensing assembly and the casing are separately joined to the enclosure mechanically and with electrical connection to the control circuitry, whereby the control circuitry receives power and/or control signals via the telemetry coil and selectively actuates the drug-dispensing assembly in accordance with a drug-delivery protocol.

26. The system of claim 25, wherein the pumping mechanism is electrolytic.
